(19) United States
(12) Patent Application Publication
(10) Pub. No.: US 2009/0035725 A1
(43) Pub. Date: Feb. 5, 2009

(54) PHOTODISINFECTION OF ORAL CAVITY

(75) Inventors: Nicholas G. Loebel, Redmond, WA (US); Cale Street, Edmonds, WA (US)

Correspondence Address: DOBRUSIN & THENNISCH PC, 29 W LAWRENCE ST, SUITE 210, PONTIAC, MI, 48342, US

(73) Assignee: Ondine International, Ltd.

(21) Appl. No.: 12/179,977

(22) Filed: Jul. 25, 2008

Related U.S. Application Data
(60) Provisional application No. 60/953,541, filed on Aug. 2, 2007.

(51) Int. Cl. A61C 17/00; A61K 8/49; A61Q 11/00; A61K 8/11

(52) U.S. Cl. 433/216; 424/401; 424/49

(57) ABSTRACT

A composition for photodisinfection of the oral cavity comprising toothpaste and a photosensitizing composition encapsulated within microspheres, wherein the microspheres release the photosensitizing composition into the oral cavity upon contact with liquid and/or force and the photosensitizing composition includes a photosensitizer that upon irradiation of at least one predetermined wavelength will reduce and/or eliminate microbes in the oral cavity. The present invention also includes a method of applying the composition into the oral cavity and applying irradiation to the oral cavity at a wavelength or wavelengths absorbed by the photosensitizing composition so as to reduce or eliminate microbes in the oral cavity.

### CLAIM OF BENEFIT OF FILING DATE

This application claims the benefit of U.S. Provisional Application Ser. No. 60/953,541 titled: “Photodisinfection of Oral Cavity” filed on Aug. 2, 2007.

### FIELD OF THE INVENTION

The present invention relates to a composition and method for photodisinfection of oral cavity. Specifically, the present invention relates to a photodisinfection composition containing toothpaste and a photosensitizing composition encapsulated in microspheres, and the use of such composition with irradiation to reduce and/or eliminate microbes in the oral cavity.

### BACKGROUND OF THE INVENTION

Chronic periodontitis, a form of inflammatory periodontal disease, is the major cause of tooth loss in adults. Patients with chronic periodontitis have inflamed pockets in the gum tissue, or gingiva, surrounding the affected tooth. Layers of bacteria build up in biofilm within these gingival pockets, leaving behind calcified accretions called calculus attached to the tooth and root surfaces. As the bacterial infection progresses, inflammatory exudates from the biofilm as well as host tissue responses can cause progressive breakdown of the hard and soft tissue structures supporting the tooth, ultimately resulting in tooth loss. Bacterial infections of the oral cavity are also gaining recognition as a source of infection in the rest of the body (e.g., bacteremias [infections of the blood], infective carditis, pulmonary disease, etc.) Such infections have also been implicated in implant rejection and may complicate the prognosis for diabetes mellitus and other autoimmune disorders. Accordingly, there is an increased desire for oral treatments that promote improved dental hygiene. It is common for consumers to look for methods beyond traditional toothpaste and mouthwash.

Photodynamic therapy for killing microbes (also known as photodisinfection) in the oral cavity was disclosed by Wilson, et al. in U.S. Pat. No. 5,611,793 and European Patent No. EP 0637976B2. These patents are herein incorporated by reference. As discussed in these patents, light in a certain wavelength and intensity range is used to illuminate a photosensitive compound that has been applied to the infected tissue(s). It is believed that the light activates the compound causing the formation of free radicals and other elements that are toxic to microbes residing in the oral cavity.

### SUMMARY OF THE INVENTION

Because photodisinfection has been shown to be effective in killing infectious microbes in the oral cavity, it would be highly desirable if it were incorporated into a daily oral care routine. The term microbes is defined herein as any and all disease-related microbes such as virus, fungus, and bacteria including Gram-negative organisms, Gram-positive organisms or the like. The present invention provides a composition for photodisinfection of the oral cavity comprising toothpaste and a photosensitizing composition encapsulated in microspheres, wherein the photosensitizing composition includes a photosensitizer that upon irradiation of at least one predetermined wavelength will reduce and/or eliminate microbes in the oral cavity. The encapsulation of the liquid photosensitizing composition in microspheres allows more efficient reduction and/or elimination of microbes in the oral cavity by generally keeping the liquid photosensitizing composition separate from the other gel and toothpaste components. The encapsulation may also provide longer shelf life. The composition of the present invention also can whiten teeth.

The present invention also provides a method of photodisinfection of the oral cavity by using the composition of the present invention in a daily or otherwise periodic tooth brushing routine and applying irradiation (e.g., light or other electromagnetic) to the oral cavity at a wavelength absorbed by the photosensitizing composition so as to reduce or eliminate microbes in the oral cavity. The photodisinfection method of the present invention can also be used for teeth whitening.

### DESCRIPTION OF THE PREFERRED EMBODIMENT

I. The Composition

The composition of the present invention includes a toothpaste and a photosensitizing composition encapsulated within microspheres, wherein the microspheres release the photosensitizing composition into the oral cavity upon contact with liquid and/or force (e.g., from tooth brushing). The toothpaste as used in this application shall mean any art-disclosed toothpaste including but not limited to toothpaste in paste and gel forms, dentifrice, or the like. Examples of toothpaste are Colgate®, Crest® and Aquafresh® brands of toothpaste. Additional examples of toothpaste can be located in www.toothpasteworld.com.

The photosensitizing composition includes at least one art-disclosed photosensitizer that has at least an antimicrobial action upon irradiation of certain wavelength(s). During the process this photosensitive compound is raised to an excited energy state, and then either passes its energy on directly to a substrate/target (type I reaction), or interacts with molecular oxygen to produce reactive oxygen species (type II reaction). While photosensitizers that have other modes of operation (e.g., generation of heat or the like) are contemplated, those types discussed above are preferred. Suitable classes of compounds that may be used as antimicrobial photosensitizers include tetrapyrroles or derivatives thereof such as porphyrins, chlorins, bacteriochlorins, phthalocyanines, naphthalocyanines, texaphyrins, verdins, purpurins or pheophorbides, phenothiazines, etc., such as those described in U.S. Pat. Nos. 6,211,335; 6,583,117; and 6,607,522 and U.S. Patent Publication 2003-0180224. Preferred phenothiazines include methylene blue (MB), toluidine blue (TBO), and those discussed in U.S. Patent publication 2004-0147508. Other preferred antimicrobial photosensitizers include indocyanine green (ICG). Combinations of two or more photosensitizers, such as MB and TBO or the like, are also suitable. For example, the at least one photosensitizer may be present in the photosensitizer composition in amounts between about 0.001 wt % and 1.000 wt %, preferably between about 0.005 wt % and about 0.5 wt %, and more preferably between about 0.01 wt % to about 0.1 wt %.

The photosensitizing composition may optionally include carriers, diluents, or other solvents for the at least one photosensitizer.

The photosensitizing composition may be any suitable phase such as a liquid, gel, paste or solid. Preferably, the composition is in a liquid phase as certain photosensitizers (e.g., methylene blue or the like) are shown to be more effective in reducing and/or eliminating microbes in a liquid form as opposed to being bound in a gel or paste.

The encapsulation of the photosensitizing composition can be performed using art-disclosed methods. For example, microencapsulation technology for use in oral hygiene care was disclosed by Norman in U.S. Pat. No. 5,403,578. The '578 patent discloses the microencapsulation of calcium peroxide, fluoride, sodium bicarbonate, or urea. U.S. Pat. No. 4,107,071 (Bayless) discloses a method for manufacture of microencapsulated particles. The disclosed microcapsules contain aqueous solutions or water-soluble solid materials. Improved shelf-life and viability result from inducing phase separation on a cross-linkable polymeric base causing the base to form a protective wall surrounding the encapsulated materials. U.S. Pat. No. 5,626,870 discloses the use of liposomes containing hydrogel particles for encapsulation. The use of hydrogel particles allows for the encapsulation of compositions with high water content and high viscosity. The hydrogel particles are enclosed in a lipid membrane and that membrane breaks down at the desired rate based on the composition of the membrane. U.S. Pat. Nos. 5,082,664; 4,235,571; and 4,089,801 discuss methods for manipulating the lipid membrane of a liposome.

The composition of the present invention may optionally include a therapeutic agent, which is any chemical, drug, medication, proteinaceous molecule, nucleic acid, lipid, antibody, antigen, hormone, nutritional supplement, cell or any combination thereof that helps ameliorate a condition. Preferred therapeutic agents include those that have antimicrobial action, have anti-inflammatory action, and/or provide pain relief.

The composition of the present invention may optionally include a teeth whitening agent such as carbamide peroxide or hydrogen peroxide. It should be noted that it is known in the arts that certain photosensitizer upon proper irradiation can whiten teeth without any additional whitening agent.

II. The Method

The present invention method for photodisinfection of the oral cavity includes applying the composition of the present invention described upon to the teeth and gum within the oral cavity (as in normal tooth brushing routine) wherein during this application process, the microspheres are disrupted and release the photosensitizing composition into the oral cavity upon contact with liquid and/or force. The method further includes applying irradiation to the oral cavity at a wavelength or wavelengths absorbed by the photosensitizing composition so as to reduce and/or eliminate microbes in the oral cavity.

The irradiation may be produced by any suitable art-disclosed light emitting devices such as lasers, light emitting diodes (“LEDs”), arc lamps, incandescent sources, fluorescent sources, or the like. Preferably, the light source is able to deliver light with a power of between about 1 J/cm² and about 20 J/cm², and more preferably between about 5 J/cm² and about 10 J/cm².

The irradiation may be at any wavelengths that can be absorbed by a photosensitizing composition. Such wavelengths include wavelengths selected from the continuous electromagnetic spectrum such as ultraviolet (“UV”), visible, the infrared (near, mid and far), etc. The wavelengths are generally between about 100 nm to about 10,000 nm, with exemplary ranges between about 160 nm to 1600 nm, between about 400 nm to about 800 nm, and between about 500 nm to about 850 nm, although the wavelengths may vary depending upon the particular photosensitizing compound used and the light intensity. In one embodiment, the light source can provide two or more wavelengths at one time or sequentially.

The method of the present invention may optionally include the application of sonic energy, preferably in a fluid (e.g., water, saliva, or the like), to the oral cavity (e.g., teeth, gum, tongue, etc.) The application of sonic energy in the oral cavity has been known in the arts to enhance dental hygiene.

As mentioned above, since the photosensitizing composition can have the ability to whiten teeth, the method of the present invention can also be used to whiten teeth.

The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the invention. The scope of the invention should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes.

What is claimed is:

1. A composition comprising:
a toothpaste; and
a photosensizing composition encapsulated in microspheres; wherein
(i) the microspheres release the photosensitizing composition into the oral cavity upon contact with liquid and/or force;
(ii) the photosensitizing composition includes a photosensitizer that upon irradiation of at least one predetermined wavelength will reduce and/or eliminate microbes in the oral cavity.

2. The composition of claim 1 wherein the microspheres are microcapsules having a polymeric membrane.

3. The composition of claim 1 wherein the microspheres are liposomes having a lipid membrane.

4. The composition of claim 1 wherein the microspheres are contacted with a liquid material suitable for application to the oral cavity.

5. The composition of claim 1 wherein the photosensitizer upon irradiation of at least one predetermined wavelength can also whiten teeth.

6. The composition of claim 1 further comprising a teeth whitening agent.

7. The composition of claim 6 wherein the teeth whitening agent is an additional photosensitizer.

8. The composition of claim 1 further comprising a therapeutic agent.

9. The composition of claim 1 wherein the photosensitizer is a phenothiazine.

10. The composition of claim 1 wherein the photosensitizer is methylene blue.

11. The composition of claim 1 wherein the photosensitizer is toluidine blue.

12. The composition of claim 1 wherein the photosensitizer is indocyanine green.

13. The composition of claim 1 wherein the concentration of the photosensitizer ranges from 0.001 wt % and 1 wt %.

14. A method for photodisinfection of the oral cavity, comprising:
applying a composition comprising a toothpaste and a photosensizing composition encapsulated in microspheres to the teeth and gum within the oral cavity, wherein the microspheres release the photosensitizing composition into the oral cavity upon contact with liquid and/or force; and
applying irradiation to the oral cavity at a wavelength absorbed by the photosensitizing composition so as to reduce or eliminate microbes in the oral cavity.

15. The method of claim 14 wherein the applying irradiation to the oral cavity step involves applying multiple wavelengths that are absorbed by the photosensitizing composition so as to reduce and/or eliminate microbes in the oral cavity.

16. The method of claim 14 wherein the wavelength ranges from about 400 nm to about 800 nm.

17. The method of claim 14 wherein the irradiation is supplied by a laser.

18. The method of claim 14 wherein the irradiation is supplied by LEDS.

19. The method of claim 14 wherein the irradiation applied has a power between about 5 J/cm² and about 10 J/cm².

20. The method of claim 14 further comprising applying sonic energy to the oral cavity.
